(19) United States
(12) Patent Application Publication
(10) Pub. No.: US 2015/0141859 A1
(43) Pub. Date: May 21, 2015

(54) SYSTEMS AND METHODS FOR CARDIAC RHYTHM VARIABILITY ANALYSIS

(71) Applicants: ALEX SANDLER, Rochester Hills, MI (US); Sergey Razhev, Nizhny Novgorod (RU); Alexander Ilyahinsky, Nizhny Novgorod (RU)

(72) Inventors: ALEX SANDLER, Rochester Hills, MI (US); Sergey Razhev, Nizhny Novgorod (RU); Alexander Ilyahinsky, Nizhny Novgorod (RU)

(21) Appl. No.: 14/084,282

(22) Filed: Nov. 19, 2013

(51) Int. Cl. A61B 5/0468; A61B 5/0245; A61B 5/0432; A61B 5/0456; A61B 5/00; A61B 5/044; A61B 5/024; A61B 5/04

(52) U.S. Cl. CPC A61B 5/0468; A61B 5/02405; A61B 5/0245; A61B 5/04012; A61B 5/0456; A61B 5/0006; A61B 5/0022; A61B 5/044; A61B 5/0432
USPC 600/516

(57) ABSTRACT

Systems and methods are provided for the study of the cardiac rhythm variability. These systems and methods include the recording of the electrocardiogram (EKG), with subsequent calculation of the duration of the beat-to-beat intervals (i.e., R-R intervals), drawing up of the rhythmograms, and additional systems and methods that include determination of, throughout all observation times within particular intervals included in the recording periods, the average values of the informational entropy of the beta-distribution (AE) of the R-R intervals, the differences between the maximum and minimum values of the informational entropy of the beta-distribution (MDE) of the R-R intervals, the root-mean-square deviations of the informational entropy of the beta-distribution (RMSDE) of the R-R intervals, and/or variation factors of the informational entropy of the beta-distribution (VFE) by means of calculation of the informational entropy of the beta-distribution of the R-R intervals.

### CROSS-REFERENCE TO RELATED APPLICATION

The instant application claims priority to U.S. Provisional Patent Application Ser. No. 61/727,967, filed Nov. 19, 2012, the entire specification of which is expressly incorporated herein by reference.

### FIELD OF THE INVENTION

The present invention relates generally to cardiac function analysis, and more specifically to new and improved systems and methods for cardiac rhythm variability analysis.

### BACKGROUND OF THE INVENTION

Cardiology and neurology, in particular, includes the study of various methods for the estimation of the vegetative regulation of cardiac rhythm. An indicator of the nervous effects on the heart is deemed to be the variability of the cardiac rhythm (“VCR”), or continuous variations of the duration of the R-R intervals of an electrocardiogram (“EKG” (also abbreviated as ECG)), which variability or variations are typically caused by continuous correction of cardiac activity according to conditions of functioning of the human organism. R-R intervals are generally defined as the time intervals between consecutive heart beats, customarily measured in the EKG from the beginning of a QRS complex to the beginning of the next QRS complex. A QRS complex is generally defined as the combination of three of the graphical deflections seen on a typical EKG. It is usually the central and most visually obvious part of the tracing. It typically corresponds to the depolarization of the right and left ventricles of the human heart. The Q, R, and S waves typically occur in rapid succession, do not all appear in all leads, and reflect a single event, and thus are usually considered together. A Q wave is any downward deflection, an R wave follows as an upward deflection, and an S wave is any downward deflection after the R wave.

Methods of analysis of the cardiac rhythm variability are also based on the recognition and measurement of the time intervals between R-surges of an EKG (i.e., RR-intervals), with the creation of dynamic series of the beat-to-beat intervals with subsequent analysis of the obtained numerical series by means of various mathematical methods. Methods of estimation of the cardiac rate variation during various time periods may be broken down into time-domain methods (e.g., see RU 2185090) and frequency-domain methods (e.g., see RU 2141246 and RU 2246251).

Both time-domain and frequency-domain methods have limitations caused by the irregularity of the series of RR-intervals. Profiles, which are clearly visible as being different from one another, may however bring about identical results when analyzed by means of these methods. Apart from this, spectral indices, due to their high variability in the course of the EKG recording, are not suitable for characterization of individual peculiarities of the human's functional state. Standard statistical parameters of the time interval, which parameters at the time of recording have stable individual peculiarities, may serve as a “portrait” of an individual person, and may allow for discerning the person's functional state from another state in conditions of relative physiological repose.

Statistical time-domain methods that have been recommended by the European Society of Cardiology and the North-American Society of Pacing and Electrophysiology as international standards are the cardiac rhythm's variability analysis methods. Statistical time-domain indices may be broken down in two primary groups: (1) obtained through processing of the direct measurements of instant cardiac rate or NN-intervals, which are time intervals between consecutive normal beats, reflecting the underlying sinus rhythm; and (2) calculated on the basis of the difference between the NN-intervals.

These indices may be calculated for all periods of observation or for some definite intervals within the period of recording. This allows for the comparison of VCR in various moments of functioning of the human organism, with variable being the most convenient to calculate, e.g., the standard deviation of the NN-intervals (“SDNN”), i.e., the square root from dispersion NN. SDNN represents all cyclic components responsible for variability within the recording period. In numerous studies, the SDNN has been calculated for all 24-hours periods, and thus includes short-term and high-frequency variations, as well as very-low-frequency components, which occur within the 24-hour period. When the recording period is shortened, the SDNN assesses more of the shorter cardiac cycles.

The total value of variability typically increases with growth of the length of the record being studied. For a randomly recorded EKG, the SDNN is not the best quantitative statistical index, due to its dependence on the length of the recording period. In practice, it is not necessarily correct to compare the SDNN values calculated from records of various length.

In statistical methods, it is also used to calculate the following indices:

SDANN, i.e., standard deviation from average values of the SDNN from 5-minutes segments for medium-duration, multiple-hours, or 24-hours records;

RMSSD, i.e., square root from the sum of squares of differences of values of the sequential pairs of the NN intervals (i.e., normal RR intervals);

NN50, i.e., the number of pairs of the sequential NN intervals differing by more than 50 milliseconds;

PNN50, i.e., the percentage of NN50 of the total number of the sequential pairs of intervals; and

CV, i.e., the variation factor, convenient for practical use, because it is a normalized estimation of the SDNN with allowance for the cardiac rate.

Apart from the aforesaid indices, statistical methods calculate: D, As, Ex, i.e., the second, third, and forth statistical moments. D, i.e., dispersion, represents the summary power of all periodic and non-periodic oscillations. As, i.e., the skewness factor, allows one to judge about stationarity of the studied dynamic series and about presence and pronouncement of the transition processes, including trends. Ex, i.e., excess factor, represents rapidity (e.g., abruptness) of the alteration of random non-stationary components of the dynamic series and reflects the presence of local non-stationarities.

Other disadvantage of the aforesaid methods of analysis of the cardiac rhythm variability consists in insufficient information of the used statistical indices. Problems of interpretation of the cardiac rhythm variability characteristics are connected with the conventional methods of mathematical data processing, e.g., when studying the character of the cardiac rate alteration in time, analyzing the events (e.g., alteration of length of the time intervals), but not the state of the vegetative regulation of human's cardiac rhythm.

One approach to the cardiac rhythm variability's estimation method included the recording of 100-200 cardiac cycles, correct ranging of the R-R intervals of the EKG, and determination of the average duration (“AD”) of the R-R intervals in the studied EKG record, variation amplitude (“VA” which is the difference between maximum and minimum values) of the R-R interval, root-mean-square deviation (“RMSD” which is the square root from the sum of squares of all deviations of the index from its average value) of the R-R intervals, modal value (“Mo” which is the most frequent value) of the R-R interval, amplitude of the modal value (“AMo” which is the percentage of the arithmetical modal values) of the R-R interval, variation factor (“VF” which is equal to 100-RMSD/AD,%) of the R-R interval, and tension index (“TI” which is equal to AMo/2VA.Mo). The aforesaid indices constitute a basis for a conclusion about the functional state of vegetative regulation of the cardiac rhythm. When doing this, it is deemed that in the state of repose, the heart is controlled by means of an autonomous regulation circuit including segmental parts of the central nervous system (i.e., the spinal cord and oblongated marrow). In the case of emotional or physical load, as well as in case of pathological tension of the regulation mechanism, cardiac activity regulation starts to involve the above segmental and subcortical structures of the central nervous system, which phenomenon is referred to as centralization of the cardiac activity. Indicators of centralization of the cardiac control are deemed to be the decrease of variation range, average duration, modal value, and root-mean-square deviation of the R-R interval, as well as an increase of the amplitude of the R-R interval's modal value and index of tension of the cardiac activity.

Thus, the main disadvantages of modern methods of cardiac function analysis include: (1) impossibility to discern, on the basis of the indices being used, functional states of patients with various cardiovascular diseases; and (2) insufficient diagnostic capability of the methods as the result of the empiric approach to the study of cardiac rhythm variability.

Accordingly, there exists a need for new and improved cardiac rhythm variability analysis systems and methods that overcome at least one of the above-mentioned disadvantages.

### SUMMARY OF THE INVENTION

One of the primary problems to be solved by the present invention is to create systems and methods for providing a high operational reliability assessment of adaptive regulatory systems of the human body by the cardiac rhythm variability.

In accordance with the general teachings of the present invention, systems and methods are provided for the study of the cardiac rhythm variability.

More specifically, these systems and methods include, without limitation, the recording of the EKG, with subsequent calculation of the duration of the beat-to-beat intervals, drawing up of the rhythmograms, and additional systems and methods that include determination of, throughout all observation times within particular intervals included in the recording periods, the average values of the informational entropy of the beta-distribution (“AE”) (wherein the phrase beta-distribution, as used herein, is meant to include a family of continuous probability distributions in probability theory and statistics defined on the interval [0, 1] parameterized by two positive shape parameters, denoted by a and β, that appear as exponents of the random variable and control the shape of the distribution), the differences between the maximum and minimum values of the informational entropy of the beta-distribution (“MDE”), the root-mean-square deviations of the informational entropy of the beta-distribution (“RMSDE”), and/or the variation factors of the informational entropy of the beta-distribution (“VFE”) by means of calculation of informational entropy of the beta-distribution for the sequence of samples nᵢ of values of beat-to-beat intervals of the rhythmogram by the formula:

$${H}_{i} = ln \frac{Γ ( {V}_{1 i} ) Γ ( {V}_{2 i} )}{Γ ( {a}_{i} )} + ( {a}_{i} - 2 ) ψ ( {a}_{i} ) - ( {V}_{1 i} - 1 ) ψ ( {V}_{1 i} ) - ( {V}_{2 i} - 1 ) ψ ( {V}_{2 i} ) ,$$

where Γ(x) corresponds to the gamma function,

where

$$ψ ( x ) = \frac{d}{d x} ln Γ ( x )$$

and corresponds to the Euler function,

where

$${v}_{1 i , 2 i} = \frac{{α}_{i}}{2} ( 1 ∓ \frac{{β}_{1 i}^{0 , 5} ( {α}_{i} + 2 )}{{[ {{β}_{1 i} ( {α}_{i} + 2 )}^{2} + 16 ( {α}_{i} + 1 ) ]}^{0 , 5}} ) , where$$

$${α}_{i} = \frac{6 ( {β}_{2 i} - {β}_{1 i} - 1 )}{6 + 3 {β}_{1 i} - 2 {β}_{2 i}} ,$$

where β₁ and β₂ skewness factor and excess index are equal to

$${β}_{1} = \frac{{μ}_{3}^{2}}{{μ}_{2}^{3}} , {β}_{2} = \frac{{μ}_{4}}{{μ}_{2}^{2}} , where$$

$${μ}_{2} = \frac{1}{( n - 1 )} {\sum }_{1}^{n} {( {x}_{j} - \overline{x} )}^{2}$$

and corresponds to a selective unbiased consistent estimation of the second central statistical moment for sample of n values of rhythmogram xⱼ,

where

$${μ}_{3} = \frac{n}{( n - 1 ) ( n - 2 )} {\sum }_{1}^{n} {( {x}_{j} - \overline{x} )}^{3}$$

and corresponds to a selective unbiased consistent estimation of the third central statistical moment for sample of n values of rhythmogram xⱼ,

where

$${μ}_{4} = \frac{( {n}^{2} - 2 n + 3 ) {\sum }_{1}^{n} {( {x}_{j} - \overline{x} )}^{4} - 3 n ( 2 n - 3 ) {( \frac{{\sum }_{1}^{n} {( {x}_{j} - \overline{x} )}^{2}}{n} )}^{2}}{( n - 1 ) ( n - 2 ) ( n - 3 )}$$

and corresponds to a selective unbiased consistent estimation of the forth central statistical moment for a sample of n values of rhythmogram xⱼ, for which x̅ corresponds to an average value of the beat-to-beat interval xⱼ,

wherein qualitative assessment of the study results may be carried out by comparison of calculated indices of informational entropy of beta-distribution with their reference values, and, in this case, any deviation of values AE, MDE, RMSDE, and/or VFE from the reference values may be deemed to be an indicator of existing pathology of the vegetative regulation of the cardiac rhythm variability.

In accordance with an aspect of the present invention, samples nᵢ for determination of the sequence of values of informational entropy of beta-distribution may be selected by a sliding shift by one number with respect to previous value of the rhythmogram's series.

In accordance with another aspect of the present invention, the volume of samples nᵢ for calculation of the sequence of values of informational entropy of beta-distribution may be selected so as it is not less than 12.

In accordance with still another aspect of the present invention, the samples nᵢ, for which αᵢ<0, do not take part in the cardiac rhythm variability analysis.

In accordance with a first embodiment of the present invention, a computer based system for cardiac rhythm variability analysis of an electrocardiogram is provided, wherein the electrocardiogram includes a plurality of recorded R-R intervals, comprising:

a computer processor unit; and

a non-transitory computer readable medium operably associated with the computer processor unit, the non-transitory computer readable medium storing instructions executable by the computer processor unit to perform the steps of:

determining an average value of an informational entropy of a beta-distribution of the plurality of R-R intervals;

determining a difference between a maximum value and a minimum value of the informational entropy of the beta-distribution of the plurality of R-R intervals;

determining a root-mean-square deviation value of the informational entropy of the beta-distribution of the plurality of R-R intervals; and

determining a variation factor percentage of the informational entropy of the beta-distribution of the plurality of R-R intervals.

In accordance with a second embodiment of the present invention, a computer implemented method for cardiac rhythm variability analysis is provided, comprising the steps of:

providing a computer processor unit;

providing a non-transitory computer readable medium operably associated with the computer processor unit, the non-transitory computer readable medium storing instructions executable by the computer processor unit to perform the steps of:

determining an average value of an informational entropy of a beta-distribution of the plurality of R-R intervals;

determining a difference between a maximum value and a minimum value of the informational entropy of the beta-distribution of the plurality of R-R intervals;

determining a root-mean-square deviation value of the informational entropy of the beta-distribution of the plurality of R-R intervals; and

determining a variation factor percentage of the informational entropy of the beta-distribution of the plurality of R-R intervals.

In accordance with a third embodiment of the present invention, a computer based system for cardiac rhythm variability analysis of an electrocardiogram is provided, wherein the electrocardiogram includes a plurality of recorded R-R intervals, comprising:

a computer processor unit;

a display device operably associated with the computer processor unit;

a data input system operably associated with the computer processor unit; and

a non-transitory computer readable medium operably associated with the computer processor unit, the non-transitory computer readable medium storing instructions executable by the computer processor unit to perform the steps of:

determining an average value of an informational entropy of a beta-distribution of the plurality of R-R intervals;

determining a difference between a maximum value and a minimum value of the informational entropy of the beta-distribution of the plurality of R-R intervals;

determining a root-mean-square deviation value of the informational entropy of the beta-distribution of the plurality of R-R intervals;

determining a variation factor percentage of the informational entropy of the beta-distribution of the plurality of R-R intervals, wherein the variation factor percentage is determined by subtracting from 100 the result of dividing the root-mean-square deviation value by the average value of the informational entropy of the beta-distribution of the plurality of R-R intervals, wherein the result of dividing the root-mean-square deviation value by the average value of the informational entropy of the beta-distribution of the plurality of R-R intervals is expressed as a positive number; and

determining a cardiac rhythm status of a patient based on any one of the steps of determining the average value of the informational entropy of the beta-distribution of the plurality of R-R intervals of the patient, determining the difference between the maximum value and the minimum value of the informational entropy of the beta-distribution of the plurality of R-R intervals of the patient, determining the root-mean-square deviation value of the informational entropy of the beta-distribution of the plurality of R-R intervals of the patient, or determining the variation factor percentage of the informational entropy of the beta-distribution of the plurality of R-R intervals of the patient.

In accordance with a fourth embodiment of the present invention, a computer implemented method for cardiac rhythm variability analysis, comprising the steps of:

providing a computer processor unit;

providing a display device operably associated with the computer processor unit;

providing a data input system operably associated with the computer processor unit; and

providing a non-transitory computer readable medium operably associated with the computer processor unit, the non-transitory computer readable medium storing instructions executable by the computer processor unit to perform the steps of:

determining an average value of an informational entropy of a beta-distribution of the plurality of R-R intervals;

determining a difference between a maximum value and a minimum value of the informational entropy of the beta-distribution of the plurality of R-R intervals;

determining a root-mean-square deviation value of the informational entropy of the beta-distribution of the plurality of R-R intervals; and

determining a variation factor percentage of the informational entropy of the beta-distribution of the plurality of R-R intervals, wherein the variation factor percentage is determined by subtracting from 100 the result of dividing the root-mean-square deviation value by the average value of the informational entropy of the beta-distribution of the plurality of R-R intervals, wherein the result of dividing the root-mean-square deviation value by the average value of the informational entropy of the beta-distribution of the plurality of R-R intervals is expressed as a positive number; and

determining a cardiac rhythm status of a patient based on any one of the steps of determining the average value of the informational entropy of the beta-distribution of the plurality of R-R intervals of the patient, determining the difference between the maximum value and the minimum value of the informational entropy of the beta-distribution of the plurality of R-R intervals of the patient, determining the root-mean-square deviation value of the informational entropy of the beta-distribution of the plurality of R-R intervals of the patient, or determining the variation factor percentage of the informational entropy of the beta-distribution of the plurality of R-R intervals of the patient.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

Other advantages of the present invention will be readily appreciated as the same becomes better understood by reference to the following detailed description when considered in connection with the accompanying drawings wherein:

FIG. 1 is a graphical illustration of the average value of AD by group of patients;

FIG. 2 is a graphical illustration of the average value of AE by group of patients;

FIG. 3 is a graphical illustration of the average value of VA by group of patients;

FIG. 4 is a graphical illustration of the average value of MDE by group of patients;

FIG. 5 is a graphical illustration of the average value of RMSD by group of patients;

FIG. 6 is a graphical illustration of the average value of RMSDE by group of patients;

FIG. 7 is a graphical illustration of the average value of VF by group of patients;

FIG. 8 is a graphical illustration of the average value of VFE by group of patients; and

FIG. 9 illustrates a schematic view an illustrative computer based system for carrying out the cardiac rhythm variability analysis of the present invention.

The same reference numerals refer to the same parts throughout the various figures.

### DETAILED DESCRIPTION OF THE INVENTION

One intended purpose of the present invention is to improve the diagnostic capability and accuracy of the estimation of the functional state of regulation systems of the human organism, and determination of the presence and/or absence of the pathology of the vegetative regulation of the cardiac rhythm.

This feature of the present invention may be attained due to the method of the study of cardiac rhythm variability including, for example, an electrocardiogram recording with the subsequent calculation of the duration of the beat-to-beat intervals and drawing up of the rhythmogram, and additionally, data on the duration of the R-R intervals according to the present invention are entered, by any means, into a computer (or other data storage/processing device), where, from the rhythmogram composed of N values of the beat-to-beat intervals, a sequence of samples of the values of the rhythmogram with volume n may be selected by means of a sliding shift by one value. For the sequence of samples nᵢ, a sequence of values of the skewness factor and the excess index may then be determined. For the sequence of values of the skewness factor and the excess index, the sequence of the values of the beta-distribution shape parameters may then be determined. For the sequence of the values of the beta-distribution shape parameters, the sequence of the values of the informational entropy of beta-distribution may then be determined by the relevant formula and then entered, by any means, into a computer memory (or other data storage medium), for example, or printed out for further study and analysis.

Estimation of the functional state of the human organism's regulatory systems, i.e., determination of the presence or absence of pathology of the vegetative regulation of the cardiac rhythm variability, may be carried out on the basis of the following parameters calculated, e.g., for all the times of observation or definite intervals within the periods of the EKG recording: (1) the average value of the informational entropy of the beta-distribution (AE); (2) the difference between the maximum and minimum values of the informational entropy of the beta-distribution (MDE); (3) the root-mean-square deviation of the informational entropy of the beta-distribution (RMSDE, i.e., the square root from the sum of squares of all deviations of an index from its average value); and/or (4) the variation factor of the informational entropy of the beta-distribution (VFE=100−(RMSDE/AE), expressed as a percentage). In other words, determining VFE includes subtracting from 100 the result of dividing RMSDE by AE, wherein the result of dividing RMSDE by AE is expressed as a positive number, even if AE is a negative number. The resulting number is then expressed as a percentage.

With this, it may be assumed that the blood circulation system is an open thermodynamic system, whose state may be determined by a set of processes running in it, such as birth/collapse processes. In case of the alteration of the cardiac rhythm due to the operation of the neuro-hormonal regulation mechanisms, the system passes along certain routes in the space of states. Assuming that a definite value of the entropy index corresponds to each such state, it is possible not only to evaluate, e.g., on the basis of the EKG record, the alterations of the state of the blood circulation system, but also to determine the presence or absence of the pathology of the vegetative regulation of the cardiac rhythm. Indicators of the pathology of the vegetative regulation of the cardiac rhythm may be deemed to be any deviation from the indices AE, MDE, RMSDE and/or VFE from their average values determined for healthy and young (from 17 to 23 y/o) persons of both sexes, whose values may be used as the VCR reference data.

Accuracy of the estimation of the statistical parameters may be determined by the number of the observation results or the volume of the empirical sample n. By way of a non-limiting example, it may be customary to assume that the greater the number of tests (e.g., volume of samples) are, the more accurate the final result of the estimation will be. The last inference is based on the use of one of the central limit theorems and the use of normal distribution for the calculation of errors. However, in this case, there may be no allowance for the fact that the increase of the volume of the sample brings about an increase of the risk of an error of measurement, especially in the case when the measurements may be conducted with the presence of disturbances, as well as brings about accumulations of errors, which forms the “noise” of the experiment-and-calculation process. Therefore, it may be necessary to strive to maximally curtail the volume of the sample of the empirical data compatible with a minimum loss of information. If an observation of the state of the object has been conducted with the presence of disturbances, then the volume of the sample n allowing to discern these two states with a confidence factor equal to 95% at the one-bit information level should be not less than 12.

In the course of implementation of the described methods, one or more of the following steps, not necessarily in the following sequence, may be carried out:

(1) From a rhythmogram with a volume N, by means of a sequential sliding shift by one number with respect to the previous value of the rhythmogram's series, a selection may be made of a sequence of samples nᵢ of values of the rhythmogram xⱼ with volume n<N;

(2) For the sequence of samples nᵢ of values of the rhythmogram xⱼ the sequence of values of the skewness factor β₁ᵢ and excess index β₂ᵢ may then be determined by the formula:

$${β}_{1} = \frac{{μ}_{3}^{2}}{{μ}_{2}^{3}} and {β}_{2} = \frac{{μ}_{4}}{{μ}_{2}^{2}} ,$$

where μ₂ corresponds to a selective unbiased consistent estimation of the second central statistical moment for the sample of n values of the rhythmogram xⱼ, and may then be calculated by the formula:

$${μ}_{2} = \frac{1}{( n - 1 )} {\sum }_{1}^{n} {( {x}_{j} - \overline{x} )}^{2} ,$$

where μ₃ corresponds to a selective unbiased consistent estimation of the third central statistical moment for the sample of n values of the rhythmogram xⱼ, and may then be calculated by the formula:

$${μ}_{3} = \frac{n}{( n - 1 ) ( n - 2 )} {\sum }_{1}^{n} {( {x}_{j} - \overline{x} )}^{3} ,$$

where μ₄ corresponds to a selective unbiased consistent estimation of the forth central statistical moment for the sample of n values of the rhythmogram xⱼ, and may then be calculated by the formula:

$${μ}_{4} = \frac{( {n}^{2} - 2 n + 3 ) {\sum }_{1}^{n} {( {x}_{j} - \overline{x} )}^{4} - 3 n ( 2 n - 3 ) {( \frac{{\sum }_{1}^{n} {( {x}_{j} - \overline{x} )}^{2}}{n} )}^{2}}{( n - 1 ) ( n - 2 ) ( n - 3 )} ,$$

where x̅ corresponds to an average value for a sample of n values of the rhythmogram xⱼ and may be calculated by the formula:

$$\overline{x} = \frac{1}{n} {\sum }_{1}^{n} {x}_{j}$$

(3) To obtain a sequence of values β₁ᵢ and β₂ᵢ i=1 . . . N−n, the sequence of the values of the shape parameters v_{1i,2i} of the beta-distribution, 0≦x≦1, v₁>0, v₂>0, α=v₁+v₂ may be calculated by the formula:

$${v}_{1 i , 2 i} = \frac{{α}_{i}}{2} ( 1 ∓ \frac{{β}_{1 i}^{0 , 5} ( {α}_{i} + 2 )}{{[ {{β}_{1 i} ( {α}_{i} + 2 )}^{2} + 16 ( {α}_{i} + 1 ) ]}^{0 , 5}} ) ,$$

where factor αᵢ may be calculated by the formula:

$${α}_{i} = \frac{6 ( {β}_{2 i} - {β}_{1 i} - 1 )}{6 + 3 {β}_{1 i} - 2 {β}_{2 i}}$$

(4) For the sequence of the values αᵢ and v_{1i,2i} the sequence of the values of the informational entropy of beta-distribution Hᵢ may then be determined by the formula:

$${H}_{i} = ln \frac{Γ ( {V}_{1 i} ) Γ ( {V}_{2 i} )}{Γ ( {a}_{i} )} + ( {a}_{i} - 2 ) ψ ( {a}_{i} ) - ( {V}_{1 i} - 1 ) ψ ( {V}_{1 i} ) - ( {V}_{2 i} - 1 ) ψ ( {V}_{2 i} ) ,$$

where Γ(x) corresponds to the gamma function, while

$$ψ ( x ) = \frac{d}{d x} ln Γ ( x )$$

and corresponds to the Euler function.

(5) Samples nᵢ, for which αᵢ<0, do not take part in analysis of the cardiac rhythm variability.

(6) Functional states of the regulating system of the human organism may be estimated on the basis of the following values calculated for all times of observation or for definite intervals within the EKG recording period: (1) the average value of the informational entropy of beta-distribution (AE); (2) the difference between the maximum and minimum values of the informational entropy of beta-distribution ((MDE); (3) the root-mean-square deviation of the informational entropy of beta-distribution (RMSDE); and/or (4) the variation factor (VFE) of the informational entropy of the beta-distribution.

(7) Deviation of the indices AE, MDE, RMSDE and VFE from their average values for healthy and young (e.g., from 17 to 23 y/o) persons of both sexes, which average values are assumed as reference VCR data, may be used as indicators of the presence or absence of the pathology of the vegetative regulation of the cardiac rhythm variability in the condition of relative physiological repose.

Considering an instance of implementation of the above-described method, as the input data a sequential record of durations (e.g., time measured in milliseconds) was taken of intervals between two neighboring RR-surges on the EKG (e.g., see column “Tᵢₙₜ” of Table 1), while the duration of this record is 5 minutes for a patient with a known diagnosis of “discirculatory encephalopathy” (e.g., see No. 1 in Table 4).

TABLE 1
Summary data of VCR analysis

| No | Tᵢₙₜ | Samples of R-R intervals (n = 12) | | | | | | | | | | | | H |
|---|---|---|---|---|---|---|---|---|---|---|---|---|---|---|
| 1 | 949 | 949 | 995 | 1055 | 944 | 898 | 940 | 958 | 947 | 897 | 922 | 949 | 944 | -1.02 |
| 2 | 995 | 995 | 1055 | 944 | 898 | 940 | 958 | 947 | 897 | 922 | 949 | 944 | 889 | -0.83 |
| 3 | 1055 | 1055 | 944 | 898 | 940 | 958 | 947 | 897 | 922 | 949 | 944 | 889 | 856 | αᵢ < 0 |
| 4 | 944 | 944 | 898 | 940 | 958 | 947 | 897 | 922 | 949 | 944 | 889 | 856 | 895 | αᵢ < 0 |
| 5 | 898 | 898 | 940 | 958 | 947 | 897 | 922 | 949 | 944 | 889 | 856 | 895 | 830 | -0.30 |
| 6 | 940 | 940 | 958 | 947 | 897 | 922 | 949 | 944 | 889 | 856 | 895 | 830 | 822 | αᵢ < 0 |
| 7 | 958 | 958 | 947 | 897 | 922 | 949 | 944 | 889 | 856 | 895 | 830 | 822 | 870 | -0.29 |
| . | . | . | . | . | . | . | . | . | . | . | . | . | . | . |
| . | . | . | . | . | . | . | . | . | . | . | . | . | . | . |
| . | . | . | . | . | . | . | . | . | . | . | . | . | . | . |
| 282 | 900 | 900 | 920 | 900 | 877 | 899 | 936 | 939 | 893 | 869 | 910 | 940 | 915 | -0.16 |
| 283 | 920 | 920 | 900 | 877 | 899 | 936 | 939 | 893 | 869 | 910 | 940 | 915 | 867 | -0.07 |
| AD | 935.55 | Msec | | | | | | | | | | | AE | -0.91 |
| VA | 253.00 | Msec | | | | | | | | | | | MDE | -22.14 |
| RMSD | 40.35 | msec² | | | | | | | | | | | RMSDE | 2.53 |
| VF | 95.68 | % | | | | | | | | | | | VFE | 97.4 |

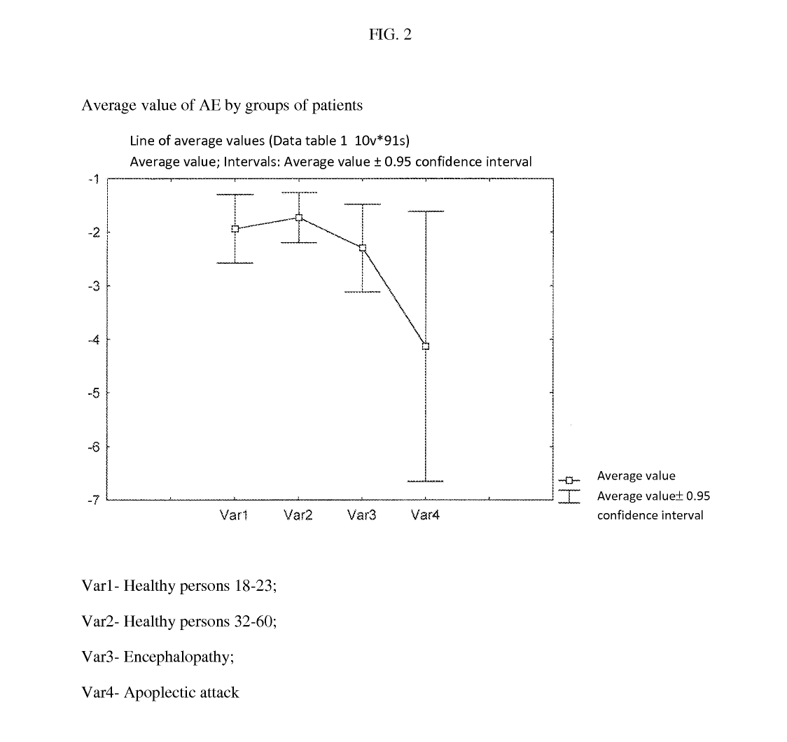

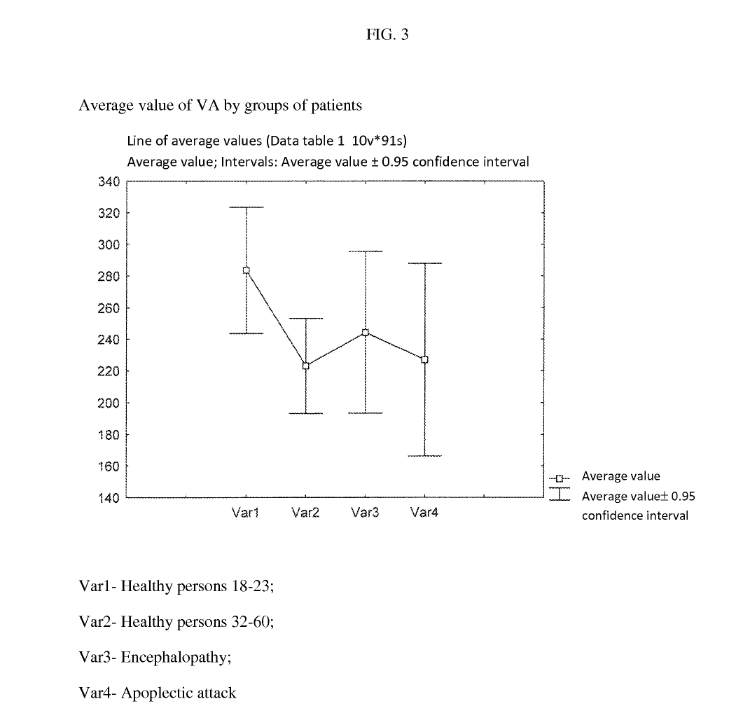

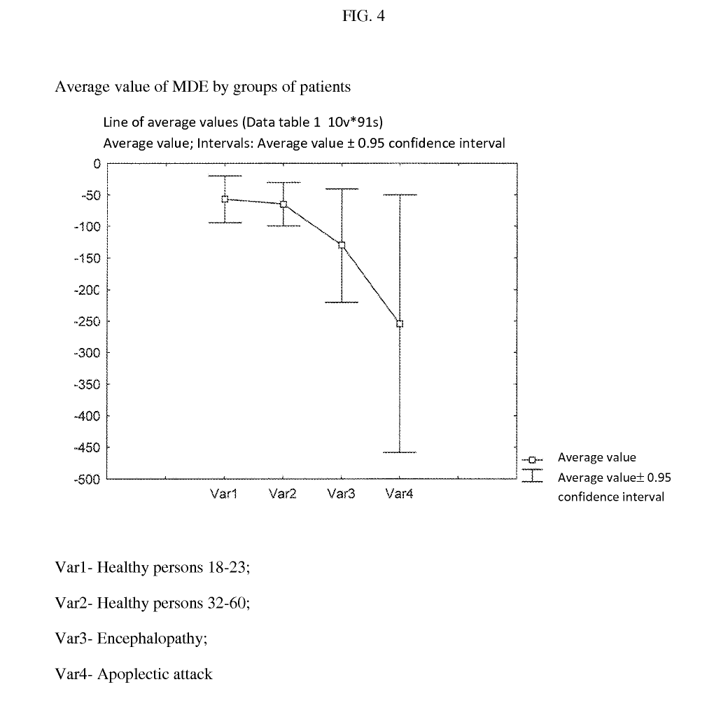

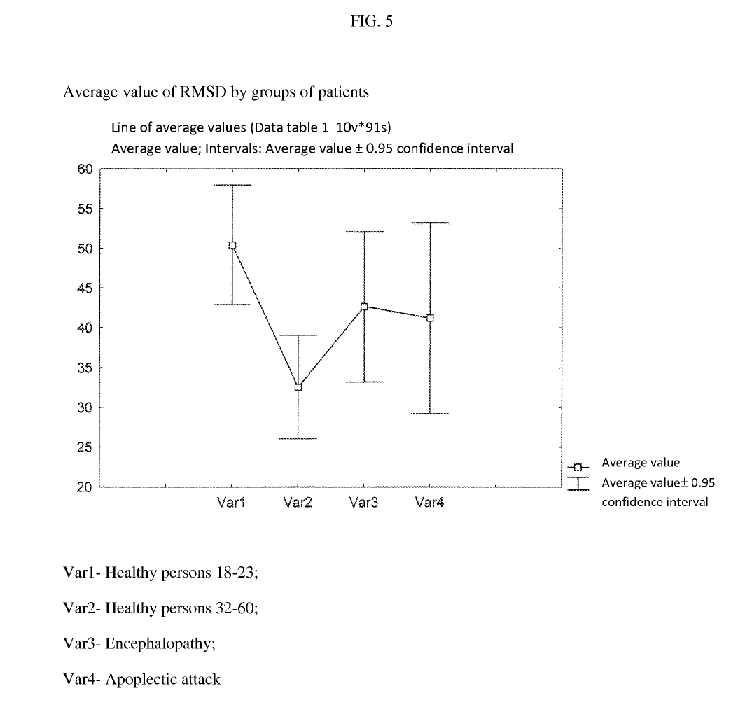

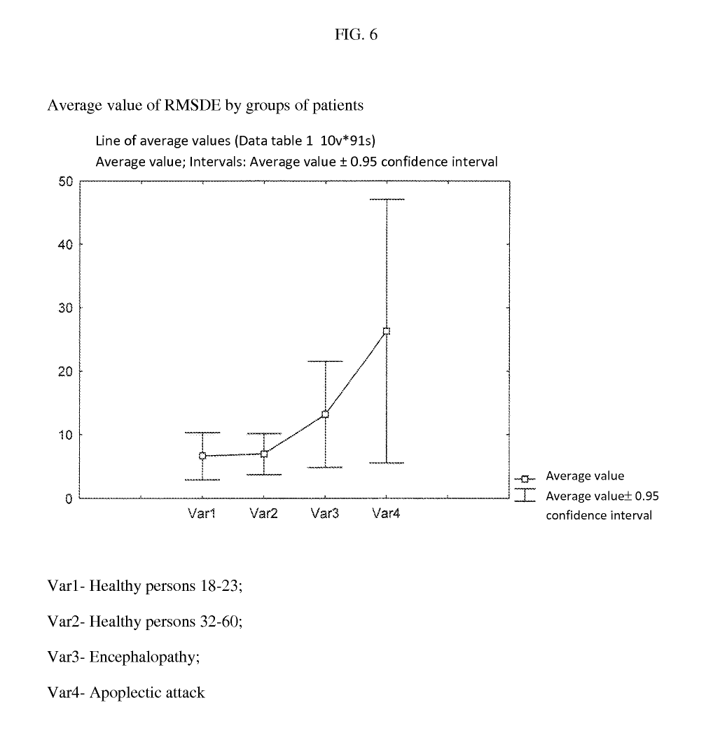

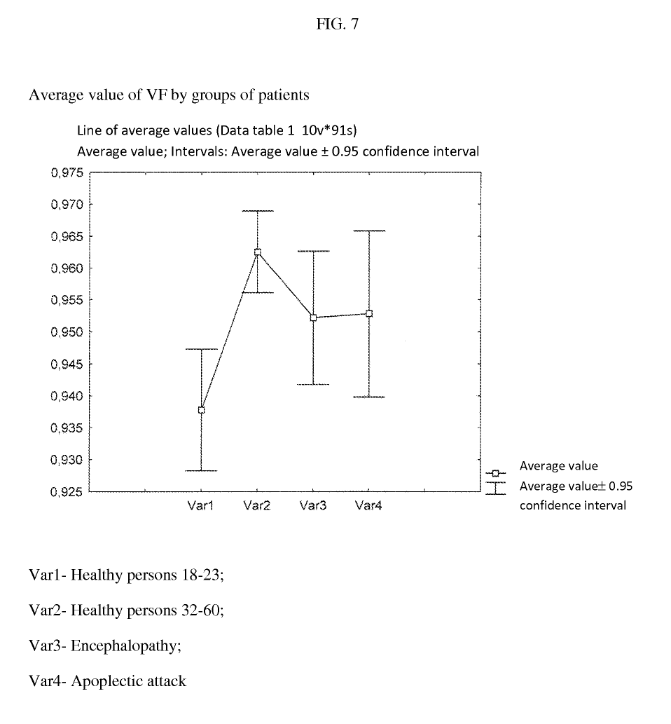

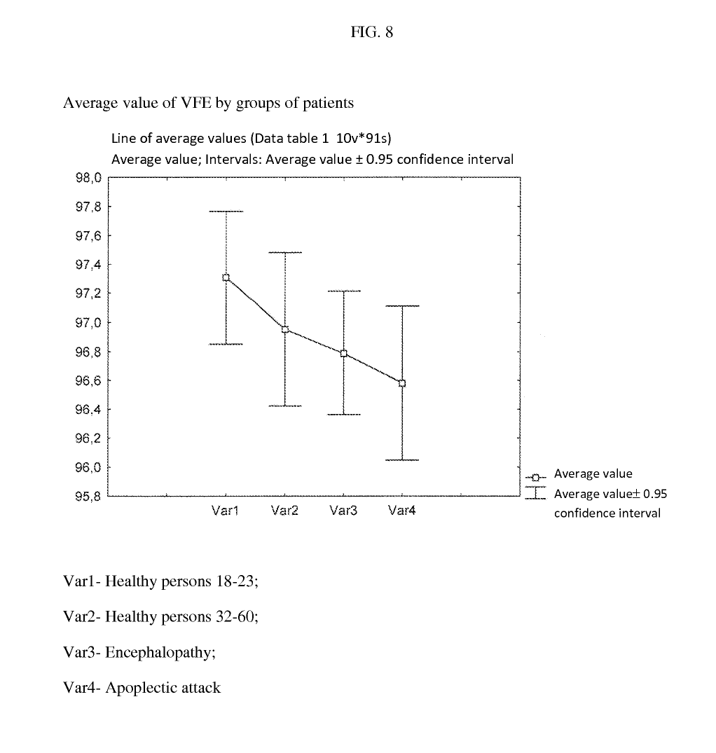

From rhythmograms with a volume equal to 283 values of the R-R intervals that were selected, by means of a sliding shift by one value with respect to previous value of the rhythmogram's series, the sequence of samples of the rhythmogram values with volume n=12 (e.g., see lines “Samples of R-R intervals” of Table 1), for which samples were determined, according to the formulas given above, the values of entropy (e.g., see column “H” of Table 1). Therefore, from analysis of the character of the variation of duration of the R-R intervals determined within the observation period by statistical values of their parameters AD=935.55 msec, VA=253 msec, RMSD=40.35 msec² and VF=95.68%, proceeding to analysis of the state of the analyzed system, in this case, of the patient, whose state within the recording period may be characterized by means of dimensionless units convenient for comparison (e.g., see Table 1) was as follows: the average value of informational entropy of beta-distribution (AE=−0.91), the difference between the maximum and minimum values of the informational entropy of the beta-distribution (MDE=−22.14); the root-mean-square deviation of the informational entropy of the beta-distribution (RMSDE=2.53); and/or the variation factor of the informational entropy of the beta-distribution (VFE=97.04). It is possible to mention, that for the patient, at the time of the EKG recording, values of the entropy indices AE, MDE, RMSDE and VFE (e.g., see FIGS. 2, 4, 6 and 8) get into the 95% confidence interval of the average values of the indices for the group “Healthy persons from 18 to 23 y/o.” Values of the prior-art index VA (e.g., see FIG. 3) also get into the 95% confidence interval of the average values of this index for the group “Healthy persons from 18 to 23 y/o,” while the values of indices RMSD and VF (e.g., see FIGS. 5 and 7) get into the interval of the average values for groups with the diagnosis of “discirculatory encephalopathy.”

In order to more particularly demonstrate the advantages of the use of the entropy parameters for evaluation of the human's functional state, the results of the classification of the patients into the groups of “Healthy persons from 18 to 23 y/o” (26 persons), “Healthy persons from to 62 y/o” (38 persons), “Patients with a diagnosis of “discirculatory encephalopathy”” (91 persons), and “Patients with a diagnosis of “apoplectic attack”” (58 persons) are presented.

Results of the calculation of the main statistical VCR parameters (e.g., prior-art parameters) and entropy parameters (e.g., present invention parameters) for rhythmograms of the above groups of patients are given in Tables 2-5, set forth below.

TABLE 2
Main statistical parameters and parameters of the
informational entropy of the beta-distribution for the group
“Healthy persons from 18 to 23 y/o.”

| | Healthy persons 18-23 y/o | | | | | | | |
|---|---|---|---|---|---|---|---|---|
| | Statistical indices | | | | Entropy parameters | | | |
| Patient | AD ms | VA ms | RMSD ms2 | VF %/100 | AE | MDE | RMSDE | VFE % |
| 1 | 771.26 | 414.00 | 65.28 | 0.92 | −1.89 | −29.50 | 4.14 | 97.81 |
| 2 | 769.48 | 138.00 | 27.34 | 0.96 | −0.60 | −15.44 | 1.52 | 97.48 |
| 3 | 608.19 | 156.00 | 21.96 | 0.96 | −6.30 | −427.21 | 41.08 | 93.48 |
| 4 | 624.40 | 201.00 | 38.68 | 0.94 | −2.27 | −91.27 | 10.06 | 95.56 |
| 5 | 699.96 | 285.00 | 63.18 | 0.91 | −2.68 | −51.16 | 6.91 | 97.42 |
| 6 | 923.13 | 383.00 | 70.45 | 0.92 | −1.94 | −57.57 | 5.94 | 96.94 |
| 7 | 674.32 | 143.00 | 24.47 | 0.96 | −1.34 | −18.73 | 2.87 | 97.86 |
| 8 | 701.34 | 249.00 | 46.17 | 0.93 | −0.86 | −11.52 | 1.63 | 98.10 |
| 9 | 996.87 | 397.00 | 66.70 | 0.93 | −0.78 | −8.84 | 1.31 | 98.31 |
| 10 | 969.96 | 345.00 | 45.93 | 0.95 | −0.59 | −5.80 | 0.85 | 98.56 |
| 11 | 1027.97 | 345.00 | 61.52 | 0.94 | −1.07 | −38.90 | 3.89 | 96.37 |
| 12 | 849.50 | 317.00 | 61.64 | 0.93 | −1.27 | −31.40 | 3.29 | 97.42 |
| 13 | 1053.58 | 261.00 | 49.56 | 0.95 | −1.84 | −37.30 | 4.49 | 97.56 |
| 14 | 932.17 | 238.00 | 51.20 | 0.95 | −2.98 | −65.42 | 8.32 | 97.21 |
| 15 | 763.75 | 302.00 | 64.23 | 0.92 | −1.19 | −19.34 | 2.45 | 97.93 |
| 16 | 735.42 | 534.00 | 104.32 | 0.86 | −6.91 | −272.97 | 30.17 | 95.63 |
| 17 | 740.31 | 293.00 | 54.18 | 0.93 | −1.45 | −32.09 | 3.57 | 97.53 |
| 18 | 878.09 | 293.00 | 50.53 | 0.94 | −0.47 | −4.38 | 0.63 | 98.65 |
| 19 | 710.58 | 359.00 | 42.90 | 0.94 | −2.30 | −63.58 | 8.39 | 96.35 |
| 20 | 753.70 | 218.00 | 41.53 | 0.94 | −0.86 | −12.38 | 1.84 | 97.85 |
| 21 | 803.58 | 103.00 | 19.44 | 0.98 | −0.92 | −10.02 | 1.54 | 98.32 |
| 22 | 822.27 | 284.00 | 45.25 | 0.94 | −2.72 | −25.90 | 4.92 | 98.19 |
| 23 | 806.87 | 217.00 | 36.61 | 0.95 | −1.00 | −21.00 | 2.73 | 97.27 |
| 24 | 811.90 | 188.00 | 31.31 | 0.96 | −1.12 | −19.56 | 2.29 | 97.95 |
| 25 | 932.12 | 347.00 | 66.55 | 0.93 | −3.68 | −87.99 | 13.08 | 96.44 |
| 26 | 764.83 | 359.00 | 58.80 | 0.92 | −1.40 | −27.31 | 3.06 | 97.81 |
| Average | 812.52 | 283.42 | 50.37 | 0.94 | −1.94 | −57.18 | 6.58 | 97.31 |
| value | | | | | | | | |

To illustrate the process of calculating VFE, consider the data from Table 2 above, specifically with reference to Patient 1. To calculate the VFE percentage for this patient, it is first necessary to calculate a value for RMDSE/AE. In this case, 2.87 is divided by −1.34. This would normally yield a result of −2.14. However, as noted above, the result of this calculation is expressed as a positive number, i.e., +2.14 in this case. This number, i.e., +2.14, is then subtracted from 100, yielding a result of 97.86, which as also noted above, is expressed as a percentage, i.e., 97.86%. Thus, the VFE percentage for Patient 1 is 97.86%. Similar calculations were performed for the other patients in this study to determine their respective VFE percentages.

TABLE 3
Main statistical parameters and parameters of the
informational entropy of the beta-distribution for the group
“Healthy persons from 30 to 62 y/o.”

| | Healthy persons 30-62 y/o | | | | | | | |
|---|---|---|---|---|---|---|---|---|
| | Statistical indices | | | | Entropy parameters | | | |
| Patient | AD ms | VA ms | RMSD ms2 | VF %/100 | AE | MDE | RMSDE | VFE % |
| 1 | 894.43 | 195.00 | 33.96 | 0.96 | −2.22 | −184.10 | 16.98 | 92.34 |
| 2 | 829.01 | 209.00 | 36.97 | 0.96 | −1.70 | −66.22 | 6.77 | 96.03 |
| 3 | 883.64 | 198.00 | 38.34 | 0.96 | −1.37 | −28.73 | 3.53 | 97.42 |
| 4 | 958.86 | 447.00 | 69.08 | 0.93 | −1.30 | −56.93 | 5.34 | 95.89 |
| 5 | 1110.65 | 247.00 | 51.46 | 0.95 | −0.90 | −19.91 | 2.19 | 97.56 |
| 6 | 965.36 | 220.00 | 43.13 | 0.96 | −0.93 | −36.92 | 3.49 | 96.24 |
| 7 | 763.10 | 153.00 | 27.61 | 0.96 | −0.67 | −4.42 | 1.01 | 98.51 |
| 8 | 1018.79 | 137.00 | 24.73 | 0.98 | −0.66 | −7.06 | 1.04 | 98.42 |
| 9 | 874.61 | 115.00 | 21.01 | 0.98 | −0.85 | −20.63 | 2.40 | 97.19 |
| 10 | 736.58 | 54.00 | 11.10 | 0.98 | −0.42 | −8.36 | 0.88 | 97.91 |
| 11 | 738.04 | 107.00 | 16.53 | 0.98 | −7.40 | −598.69 | 55.91 | 92.45 |
| 12 | 909.83 | 103.00 | 21.22 | 0.98 | −3.16 | −134.39 | 14.86 | 95.29 |
| 13 | 853.42 | 136.00 | 24.03 | 0.97 | −1.89 | −67.68 | 7.14 | 96.23 |
| 14 | 944.64 | 112.00 | 21.23 | 0.98 | −0.42 | −2.97 | 0.45 | 98.94 |
| 15 | 693.90 | 39.00 | 8.71 | 0.99 | −2.04 | −30.35 | 4.65 | 97.72 |
| 16 | 964.25 | 146.00 | 28.87 | 0.97 | −2.77 | −162.00 | 15.64 | 94.35 |
| 17 | 1173.03 | 278.00 | 36.93 | 0.97 | −1.08 | −21.26 | 2.47 | 97.72 |
| 18 | 798.81 | 169.00 | 31.35 | 0.96 | −1.16 | −29.22 | 3.28 | 97.17 |
| 19 | 805.98 | 239.00 | 42.32 | 0.95 | −1.06 | −23.18 | 3.02 | 97.14 |
| 20 | 1115.97 | 431.00 | 62.93 | 0.94 | −1.58 | −62.82 | 6.10 | 96.13 |
| 21 | 793.86 | 93.00 | 18.46 | 0.98 | −2.47 | −131.49 | 13.12 | 94.68 |
| 22 | 563.90 | 78.00 | 18.29 | 0.97 | −4.20 | −117.27 | 13.86 | 96.70 |
| 23 | 826.14 | 73.00 | 12.57 | 0.98 | −0.71 | −5.54 | 0.95 | 98.66 |
| 24 | 1057.90 | 548.00 | 102.95 | 0.90 | −1.56 | −22.43 | 3.16 | 97.97 |
| 25 | 805.91 | 407.00 | 66.23 | 0.92 | −2.18 | −50.77 | 6.74 | 96.91 |
| 26 | 716.80 | 139.00 | 23.50 | 0.97 | −0.89 | −9.02 | 1.59 | 98.21 |
| 27 | 676.72 | 97.00 | 19.20 | 0.97 | −1.99 | −48.61 | 6.09 | 96.94 |
| 28 | 695.52 | 108.00 | 15.99 | 0.98 | −1.42 | −43.49 | 4.43 | 96.87 |
| 29 | 830.44 | 102.00 | 17.29 | 0.98 | −2.14 | −87.49 | 8.88 | 95.85 |
| 30 | 825.50 | 73.00 | 14.74 | 0.98 | −0.31 | −2.01 | 0.34 | 98.88 |
| 31 | 821.86 | 173.00 | 34.82 | 0.96 | −0.85 | −4.86 | 0.92 | 98.92 |
| 32 | 839.66 | 240.00 | 44.99 | 0.95 | −0.60 | −4.21 | 0.73 | 98.79 |
| 33 | 802.62 | 125.00 | 25.11 | 0.97 | −1.82 | −23.48 | 3.59 | 98.03 |
| 34 | 683.25 | 100.00 | 19.05 | 0.97 | −2.23 | −67.01 | 8.40 | 96.23 |
| 35 | 691.14 | 75.00 | 14.16 | 0.98 | −0.78 | −7.31 | 1.28 | 98.35 |
| 36 | 717.69 | 184.00 | 26.74 | 0.96 | −1.09 | −21.87 | 2.36 | 97.84 |
| 37 | 807.35 | 252.00 | 56.26 | 0.93 | −5.74 | −248.04 | 25.44 | 95.57 |
| 38 | 849.72 | 303.00 | 54.28 | 0.94 | −1.22 | −16.79 | 2.43 | 98.02 |
| Average | 843.13 | 181.71 | 32.53 | 0.96 | −1.73 | −65.20 | 6.88 | 96.95 |
| value | | | | | | | | |

TABLE 4
Main statistical parameters and parameters of the
informational entropy of the beta-distribution for the group
“Patients with a diagnosis of “discirculatory encephalopathy.””

| | Discirculatory encephalopathy | | | | | | | |
|---|---|---|---|---|---|---|---|---|
| | Statistical indices | | | | Entropy parameters | | | |
| Patient | AD ms | VA ms | RMSD ms2 | VF %/100 | AE | MDE | RMSDE | VFE % |
| 1 | 935.55 | 253.00 | 40.35 | 0.96 | −0.91 | −22.14 | 2.53 | 97.04 |
| 2 | 1051.56 | 285.00 | 39.28 | 0.96 | −4.07 | −189.55 | 20.02 | 95.08 |
| 3 | 1034.06 | 136.00 | 19.90 | 0.98 | −1.24 | −16.50 | 2.41 | 98.06 |
| 4 | 713.80 | 163.00 | 29.49 | 0.96 | −2.10 | −61.74 | 6.91 | 96.71 |
| 5 | 682.64 | 74.00 | 15.17 | 0.98 | −1.18 | −16.80 | 2.19 | 98.14 |
| 6 | 994.55 | 235.00 | 46.06 | 0.95 | −0.75 | −14.24 | 1.66 | 97.77 |
| 7 | 1060.31 | 240.00 | 45.91 | 0.96 | −1.14 | −27.96 | 3.31 | 97.10 |
| 8 | 822.19 | 68.00 | 12.92 | 0.98 | −3.19 | −112.92 | 12.62 | 96.05 |
| 9 | 818.78 | 75.00 | 14.28 | 0.98 | −0.46 | −10.47 | 1.04 | 97.74 |
| 10 | 563.64 | 710.00 | 121.21 | 0.78 | −3.42 | −55.00 | 8.30 | 97.58 |
| 11 | 791.54 | 222.00 | 39.20 | 0.95 | −1.28 | −65.95 | 6.30 | 95.08 |
| 12 | 725.30 | 269.00 | 46.33 | 0.94 | −1.55 | −31.38 | 4.09 | 97.35 |
| 13 | 906.94 | 285.00 | 50.78 | 0.94 | −0.80 | −7.25 | 1.22 | 98.48 |
| 14 | 725.76 | 92.00 | 19.26 | 0.97 | −2.78 | −36.38 | 6.26 | 97.75 |
| 15 | 663.95 | 82.00 | 14.51 | 0.98 | −0.59 | −11.16 | 1.33 | 97.76 |
| 16 | 823.59 | 157.00 | 21.80 | 0.97 | −1.31 | −46.84 | 4.77 | 96.35 |
| 17 | 852.63 | 253.00 | 38.95 | 0.95 | −0.79 | −19.28 | 2.00 | 97.48 |
| 18 | 1232.81 | 986.00 | 111.97 | 0.91 | −1.27 | −16.84 | 2.56 | 97.98 |
| 19 | 968.80 | 163.00 | 16.34 | 0.98 | −1.88 | −64.65 | 6.37 | 96.60 |
| 20 | 617.09 | 97.00 | 16.60 | 0.97 | −1.26 | −28.13 | 3.36 | 97.34 |
| 21 | 795.43 | 167.00 | 26.19 | 0.97 | −1.45 | −36.45 | 4.94 | 96.59 |
| 22 | 871.85 | 219.00 | 44.75 | 0.95 | −0.60 | −11.78 | 1.28 | 97.87 |
| 23 | 925.16 | 125.00 | 19.67 | 0.98 | −0.54 | −4.81 | 0.77 | 98.57 |
| 24 | 948.72 | 101.00 | 20.02 | 0.98 | −1.33 | −63.39 | 5.98 | 95.51 |
| 25 | 794.54 | 131.00 | 24.88 | 0.97 | −0.53 | −5.92 | 0.80 | 98.50 |
| 26 | 957.40 | 140.00 | 26.16 | 0.97 | −0.72 | −10.42 | 1.27 | 98.23 |
| 27 | 986.91 | 232.00 | 32.09 | 0.97 | −1.05 | −8.03 | 1.65 | 98.42 |
| 28 | 931.01 | 217.00 | 30.15 | 0.97 | −1.42 | −14.75 | 2.73 | 98.08 |
| 29 | 874.54 | 199.00 | 35.48 | 0.96 | −0.98 | −12.91 | 1.67 | 98.29 |
| 30 | 810.52 | 167.00 | 23.02 | 0.97 | −0.58 | −5.33 | 0.93 | 98.39 |
| 31 | 908.08 | 284.00 | 44.46 | 0.95 | −0.75 | −19.97 | 2.35 | 96.86 |
| 32 | 1110.52 | 170.00 | 16.12 | 0.99 | −0.87 | −10.67 | 1.70 | 98.04 |
| 33 | 945.00 | 191.00 | 38.18 | 0.96 | −2.05 | −131.61 | 12.21 | 94.03 |
| 34 | 987.46 | 1644.00 | 290.00 | 0.71 | −1.45 | −23.42 | 2.87 | 98.02 |
| 35 | 833.06 | 1047.00 | 200.75 | 0.76 | −1.39 | −75.69 | 7.12 | 94.89 |
| 36 | 856.78 | 81.00 | 16.28 | 0.98 | −0.35 | −2.75 | 0.43 | 98.79 |
| 37 | 1031.74 | 214.00 | 47.58 | 0.95 | −0.64 | −8.45 | 1.19 | 98.15 |
| 38 | 1066.52 | 336.00 | 67.87 | 0.94 | −23.87 | −2656.33 | 245.48 | 89.72 |
| 39 | 955.46 | 393.00 | 95.08 | 0.90 | −1.39 | −15.82 | 2.86 | 97.95 |
| 40 | 1020.62 | 275.00 | 42.99 | 0.96 | −0.71 | −18.99 | 1.91 | 97.32 |
| 41 | 975.63 | 154.00 | 26.90 | 0.97 | −0.93 | −43.63 | 4.08 | 95.63 |
| 42 | 948.14 | 181.00 | 33.45 | 0.96 | −0.58 | −5.19 | 0.81 | 98.59 |
| 43 | 907.90 | 254.00 | 32.39 | 0.96 | −0.72 | −5.34 | 0.95 | 98.69 |
| 44 | 810.92 | 138.00 | 23.79 | 0.97 | −3.38 | −321.62 | 29.69 | 91.21 |
| 45 | 877.79 | 92.00 | 15.18 | 0.98 | −0.31 | −3.04 | 0.45 | 98.57 |
| 46 | 976.08 | 179.00 | 30.29 | 0.97 | −1.28 | −43.44 | 5.50 | 95.71 |
| 47 | 866.58 | 121.00 | 23.99 | 0.97 | −1.03 | −38.70 | 3.64 | 96.47 |
| 48 | 907.83 | 135.00 | 23.60 | 0.97 | −0.42 | −4.17 | 0.64 | 98.48 |
| 49 | 805.96 | 388.00 | 42.13 | 0.95 | −1.68 | −43.06 | 5.24 | 96.89 |
| 50 | 805.72 | 103.00 | 14.50 | 0.98 | −0.64 | −5.54 | 0.84 | 98.68 |
| 51 | 1042.85 | 491.00 | 99.55 | 0.90 | −8.06 | −251.22 | 32.21 | 96.00 |
| 52 | 1004.74 | 450.00 | 78.33 | 0.92 | −1.01 | −10.96 | 1.77 | 98.24 |
| 53 | 698.52 | 240.00 | 39.19 | 0.94 | −1.89 | −69.17 | 6.72 | 96.44 |
| 54 | 973.78 | 971.00 | 171.68 | 0.82 | −4.62 | −84.43 | 13.11 | 97.16 |
| 55 | 984.26 | 286.00 | 36.01 | 0.96 | −0.67 | −4.84 | 0.76 | 98.86 |
| 56 | 925.37 | 317.00 | 61.83 | 0.93 | −1.82 | −71.84 | 8.24 | 95.48 |
| 57 | 1121.54 | 177.00 | 25.91 | 0.98 | −0.88 | −10.64 | 1.42 | 98.40 |
| 58 | 912.65 | 189.00 | 34.14 | 0.96 | −4.70 | −373.00 | 35.06 | 92.53 |
| 59 | 766.37 | 196.00 | 40.51 | 0.95 | −1.25 | −20.94 | 3.19 | 97.45 |
| 60 | 809.13 | 219.00 | 47.50 | 0.94 | −3.29 | −112.55 | 15.40 | 95.32 |
| 61 | 700.30 | 113.00 | 20.60 | 0.97 | −3.22 | −253.29 | 23.17 | 92.80 |
| 62 | 797.50 | 73.00 | 13.76 | 0.98 | −1.30 | −54.63 | 5.13 | 96.06 |
| 63 | 690.08 | 100.00 | 16.73 | 0.98 | −0.95 | −16.97 | 1.95 | 97.95 |
| 64 | 872.32 | 493.00 | 61.65 | 0.93 | −3.65 | −214.11 | 20.44 | 94.40 |
| 65 | 806.38 | 70.00 | 15.82 | 0.98 | −0.94 | −13.18 | 2.15 | 97.70 |
| 66 | 707.70 | 124.00 | 22.69 | 0.97 | −0.66 | −13.43 | 1.48 | 97.76 |
| 67 | 740.52 | 110.00 | 20.61 | 0.97 | −1.24 | −54.52 | 5.33 | 95.70 |
| 68 | 1026.37 | 157.00 | 23.52 | 0.98 | −2.54 | −42.16 | 6.76 | 97.33 |
| 69 | 726.22 | 56.00 | 11.53 | 0.98 | −0.57 | −3.15 | 0.72 | 98.74 |
| 70 | 1056.48 | 200.00 | 35.82 | 0.97 | −17.87 | −1977.59 | 182.75 | 89.77 |
| 71 | 699.21 | 722.00 | 144.11 | 0.79 | −1.20 | −11.95 | 1.72 | 98.57 |
| 72 | 989.73 | 259.00 | 45.46 | 0.95 | −0.57 | −5.02 | 0.84 | 98.52 |
| 73 | 823.39 | 108.00 | 20.22 | 0.98 | −2.61 | −135.30 | 13.41 | 94.86 |
| 74 | 977.64 | 86.00 | 17.25 | 0.98 | −0.94 | −14.04 | 1.68 | 98.21 |
| 75 | 851.14 | 109.00 | 21.93 | 0.97 | −23.93 | −2586.75 | 244.35 | 89.79 |
| 76 | 932.54 | 182.00 | 26.25 | 0.97 | −4.34 | −163.16 | 21.16 | 95.12 |
| 77 | 834.04 | 86.00 | 18.14 | 0.98 | −1.72 | −32.38 | 3.96 | 97.70 |
| 78 | 1118.04 | 100.00 | 16.59 | 0.99 | −0.67 | −8.06 | 1.06 | 98.41 |
| 79 | 715.01 | 108.00 | 17.23 | 0.98 | −2.97 | −82.77 | 9.21 | 96.90 |
| 80 | 1285.10 | 274.00 | 33.73 | 0.97 | −2.72 | −154.15 | 15.43 | 94.32 |
| 81 | 796.37 | 114.00 | 18.87 | 0.98 | −0.73 | −14.31 | 1.72 | 97.64 |
| 82 | 870.34 | 246.00 | 43.43 | 0.95 | −1.96 | −145.39 | 13.32 | 93.19 |
| 83 | 1093.91 | 676.00 | 197.64 | 0.82 | −9.18 | −125.79 | 19.80 | 97.84 |
| 84 | 838.47 | 285.00 | 52.72 | 0.94 | −1.45 | −57.38 | 5.61 | 96.13 |
| 85 | 1002.62 | 93.00 | 20.14 | 0.98 | −0.80 | −10.93 | 1.32 | 98.35 |
| 86 | 642.63 | 98.00 | 14.84 | 0.98 | −1.02 | −15.31 | 1.81 | 98.22 |
| 87 | 1035.72 | 161.00 | 25.97 | 0.97 | −2.15 | −56.48 | 6.84 | 96.82 |
| 88 | 953.95 | 175.00 | 35.58 | 0.96 | −0.82 | −11.89 | 1.60 | 98.03 |
| 89 | 1024.96 | 156.00 | 28.57 | 0.97 | −0.52 | −11.89 | 1.20 | 97.67 |
| 90 | 745.71 | 22.00 | 4.56 | 0.99 | −0.67 | −9.93 | 1.35 | 97.97 |
| 91 | 890.41 | 198.00 | 29.52 | 0.97 | −1.87 | −79.22 | 10.21 | 94.54 |
| Average | 889.71 | 244.10 | 42.62 | 0.95 | −2.30 | −130.67 | 13.15 | 96.79 |
| value | | | | | | | | |

TABLE 5
Main statistical parameters and parameters of the
informational entropy of the beta-distribution for the group
“Patients with a diagnosis of “apoplectic attack.””

| | Apoplectic attack | | | | | | | |
|---|---|---|---|---|---|---|---|---|
| | Statistical indices | | | | Entropy parameters | | | |
| Patient | AD ms | VA ms | RMSD ms2 | VF %/100 | AE | MDE | RMSDE | VFE % |
| 1 | 781.67 | 98.00 | 17.19 | 0.98 | −0.79 | −9.17 | 1.19 | 98.48 |
| 2 | 940.95 | 443.00 | 75.46 | 0.92 | −3.79 | −268.18 | 25.52 | 93.26 |
| 3 | 647.63 | 40.00 | 8.12 | 0.99 | −3.09 | −142.26 | 14.61 | 95.27 |
| 4 | 655.90 | 66.00 | 12.33 | 0.98 | −1.72 | −46.42 | 5.41 | 96.86 |
| 5 | 747.00 | 477.00 | 94.26 | 0.87 | −3.97 | −44.62 | 6.70 | 98.31 |
| 6 | 767.71 | 243.00 | 45.29 | 0.94 | −3.47 | −204.42 | 22.03 | 93.65 |
| 7 | 716.04 | 42.00 | 9.10 | 0.99 | −3.37 | −150.54 | 15.13 | 95.51 |
| 8 | 1082.96 | 207.00 | 33.06 | 0.97 | −1.22 | −40.22 | 3.89 | 96.80 |
| 9 | 640.84 | 130.00 | 32.30 | 0.95 | −2.20 | −99.03 | 9.28 | 95.78 |
| 10 | 805.22 | 204.00 | 36.39 | 0.95 | −3.76 | −121.94 | 12.96 | 96.56 |
| 11 | 848.10 | 61.00 | 13.94 | 0.98 | −10.17 | −418.84 | 48.41 | 95.24 |
| 12 | 877.77 | 61.00 | 9.95 | 0.99 | −1.33 | −36.91 | 4.21 | 96.84 |
| 13 | 751.83 | 181.00 | 34.82 | 0.95 | −1.18 | −22.77 | 3.27 | 97.23 |
| 14 | 759.52 | 116.00 | 18.29 | 0.98 | −0.86 | −23.47 | 2.32 | 97.31 |
| 15 | 965.07 | 259.00 | 48.00 | 0.95 | −0.58 | −4.08 | 0.82 | 98.60 |
| 16 | 974.29 | 295.00 | 42.56 | 0.96 | −3.09 | −114.07 | 14.58 | 95.28 |
| 17 | 834.91 | 90.00 | 18.14 | 0.98 | −0.97 | −13.28 | 1.70 | 98.23 |
| 18 | 1044.35 | 290.00 | 43.64 | 0.96 | −6.71 | −376.94 | 37.88 | 94.35 |
| 19 | 846.65 | 1014.00 | 219.43 | 0.74 | −2.23 | −37.84 | 5.18 | 97.68 |
| 20 | 778.48 | 52.00 | 8.03 | 0.99 | −32.35 | −3530.06 | 333.48 | 89.69 |
| 21 | 874.31 | 111.00 | 18.42 | 0.98 | −1.34 | −38.26 | 4.23 | 96.83 |
| 22 | 884.46 | 119.00 | 22.57 | 0.97 | −0.44 | −4.34 | 0.70 | 98.41 |
| 23 | 865.38 | 153.00 | 24.26 | 0.97 | −0.77 | −7.06 | 1.03 | 98.67 |
| 24 | 858.11 | 246.00 | 48.43 | 0.94 | −1.79 | −52.97 | 5.79 | 96.77 |
| 25 | 764.97 | 100.00 | 15.63 | 0.98 | −3.98 | −60.24 | 8.15 | 97.95 |
| 26 | 757.87 | 781.00 | 142.43 | 0.81 | −1.14 | −14.90 | 1.93 | 98.30 |
| 27 | 839.65 | 242.00 | 50.66 | 0.94 | −0.76 | −5.81 | 0.94 | 98.76 |
| 28 | 1028.89 | 179.00 | 26.78 | 0.97 | −1.90 | −56.34 | 6.91 | 96.37 |
| 29 | 848.69 | 84.00 | 14.89 | 0.98 | −2.16 | −51.11 | 6.46 | 97.01 |
| 30 | 647.52 | 53.00 | 9.97 | 0.98 | −6.72 | −665.79 | 60.74 | 90.96 |
| 31 | 835.36 | 207.00 | 27.83 | 0.97 | −1.94 | −26.95 | 4.23 | 97.81 |
| 32 | 751.22 | 126.00 | 28.41 | 0.96 | −1.23 | −26.57 | 3.26 | 97.35 |
| 33 | 829.10 | 83.00 | 16.47 | 0.98 | −0.68 | −10.73 | 1.51 | 97.77 |
| 34 | 842.43 | 801.00 | 141.58 | 0.83 | −1.75 | −57.01 | 5.75 | 96.72 |
| 35 | 712.10 | 33.00 | 5.93 | 0.99 | −1.46 | −54.59 | 5.51 | 96.23 |
| 36 | 788.60 | 141.00 | 24.84 | 0.97 | −5.43 | −302.45 | 30.50 | 94.38 |
| 37 | 895.60 | 180.00 | 27.79 | 0.97 | −2.86 | −30.99 | 5.81 | 97.97 |
| 38 | 917.65 | 120.00 | 25.92 | 0.97 | −0.50 | −4.78 | 0.71 | 98.60 |
| 39 | 742.81 | 86.00 | 17.32 | 0.98 | −0.95 | −42.09 | 3.91 | 95.86 |
| 40 | 882.56 | 94.00 | 15.98 | 0.98 | −0.95 | −20.26 | 2.17 | 97.71 |
| 41 | 682.17 | 87.00 | 13.55 | 0.98 | −1.00 | −37.44 | 3.62 | 96.39 |
| 42 | 721.06 | 103.00 | 19.52 | 0.97 | −1.67 | −23.57 | 3.77 | 97.73 |
| 43 | 771.14 | 162.00 | 31.33 | 0.96 | −2.06 | −47.97 | 5.48 | 97.35 |
| 44 | 897.14 | 93.00 | 23.35 | 0.97 | −1.37 | −25.93 | 2.89 | 97.89 |
| 45 | 864.03 | 400.00 | 73.24 | 0.92 | −1.87 | −37.76 | 5.28 | 97.17 |
| 46 | 985.07 | 198.00 | 28.95 | 0.97 | −31.92 | −3191.95 | 299.05 | 90.63 |
| 47 | 928.96 | 555.00 | 79.85 | 0.91 | −61.27 | −3786.44 | 429.73 | 92.99 |
| 48 | 761.19 | 133.00 | 25.07 | 0.97 | −0.85 | −12.75 | 1.79 | 97.88 |
| 49 | 864.95 | 137.00 | 24.77 | 0.97 | −1.15 | −26.15 | 3.09 | 97.32 |
| 50 | 853.77 | 53.00 | 10.18 | 0.99 | −0.74 | −14.97 | 1.82 | 97.53 |
| 51 | 814.38 | 161.00 | 28.63 | 0.96 | −0.45 | −3.52 | 0.54 | 98.78 |
| 52 | 769.04 | 147.00 | 23.68 | 0.97 | −0.99 | −7.96 | 1.58 | 98.40 |
| 53 | 950.24 | 255.00 | 46.59 | 0.95 | −1.91 | −65.99 | 7.41 | 96.12 |
| 54 | 992.31 | 264.00 | 43.91 | 0.96 | −2.44 | −117.83 | 11.34 | 95.36 |
| 55 | 1094.15 | 320.00 | 49.41 | 0.95 | −1.00 | −10.37 | 1.71 | 98.30 |
| 56 | 895.00 | 220.00 | 42.57 | 0.95 | −1.52 | −67.31 | 6.46 | 95.74 |
| 57 | 1114.47 | 1247.00 | 251.95 | 0.77 | −1.34 | −53.15 | 5.10 | 96.19 |
| 58 | 1007.71 | 332.00 | 52.28 | 0.95 | −0.79 | −8.16 | 1.31 | 98.35 |
| Average | 844.84 | 227.16 | 41.30 | 0.95 | −4.14 | −254.78 | 26.22 | 96.58 |
| value | | | | | | | | |

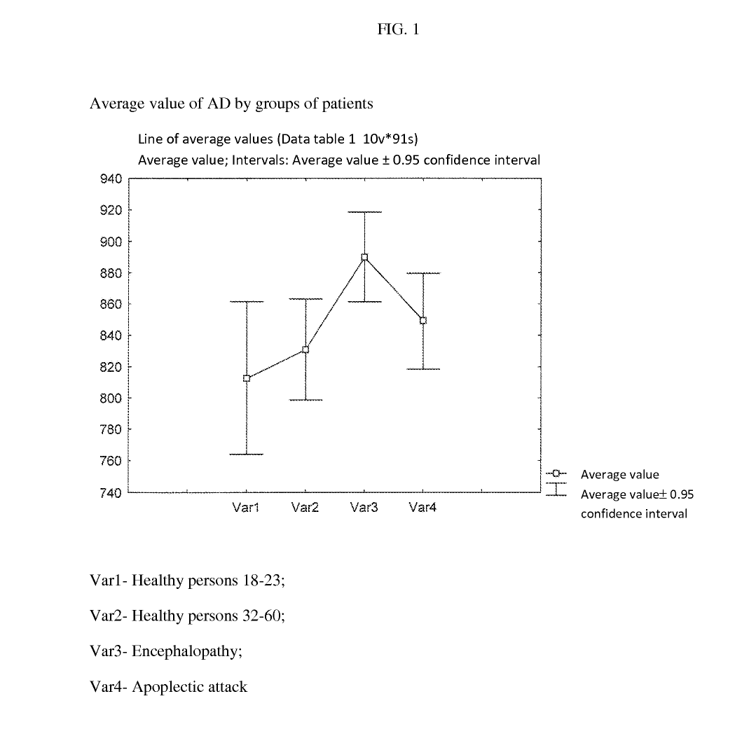

FIGS. 1-8 show the lines of average values of the main prior art statistical parameters and the present invention's parameters of the informational entropy of the beta-distribution by groups of patients for “Healthy persons from 18 to 23 y/o,” “Healthy persons from 30 to 62 y/o,” “Patients with a diagnosis of “discirculatory encephalopathy,”” and “Patients with a diagnosis of “apoplectic attack,”” which plot lines were drawn using STATISTIKA™ software for data taken from Tables 2-5.

As it is possible to see from the Figures, the transition from traditional parameters of the description of the VCR to parameters of the informational entropy of the beta-distribution allows one to trace more clearly the trend of transition from the healthy persons 18-23 y/o state of the cardiovascular system, which may be assumed as a reference, to that of the aged and diseased persons.

The results of the classification of the patients into the groups “healthy persons”/“diseased persons” are presented below.

As the “healthy” group, a joint group of patients was selected that was composed of 64 “healthy persons from 18 to 23 y/o” and “healthy persons from 30 to 62 y/o.” As the “diseased” group, a joint group of patients was selected that was composed of 149 “patients with a diagnosis of “discirculatory encephalopathy”” and “patients with a diagnosis of “apoplectic attack.””

The classification was carried out by standard statistical parameters having stable individual properties and serving as a “portrait” of a particular patient and allowing one to discern this patient from another one in an initial state of repose, i.e., the prior-art parameters, being:

(1) average duration (AD) of R-R interval in the studied EKG record;

(2) variation amplitude (VA—difference between the maximum and minimum values) of the R-R intervals;

(3) root-mean-square deviation (RMSD—the square root from the sum of squares of all deviations of an index from its average value) of the R-R intervals; and

(4) variation factor (VF=100−RMSD/AD,%) of the R-R interval,

and by parameters of state (e.g., entropy), i.e., the parameters of the described method of the present invention being:

(1) average value of the informational entropy of the beta-distribution (AE) in the studied EKG record;

(2) difference between the maximum and minimum values of the informational entropy of the beta-distribution (MDE) in the studied EKG record;

(3) root-mean-square deviation of the informational entropy of the beta-distribution (RMSDE) in the studied EKG record; and

(4) variation factor of the informational entropy of the beta-distribution (VFE) in the studied EKG record.

Accuracy of the classification may be estimated by means of a neural network composed of 2-layer perceptron with a number of neurons by layers: e.g., 1^{st} layer, i.e., 17 neurons, and 2^{nd} layer, i.e., 14 neurons, using the cross-validation method with the following breakdown of the input data into the learning and test samples: 10 elements, i.e., learning sample, and the rest of the elements, i.e., test sample.

As the function of the activation of the neuron, the following linear function may be used:

f(s)=k*s+d

where k, d are tuning parameters of the neuron. Learning of the neural network consisted in minimization of the error functional over (0:

Q(ω)=½Σᵢ₌₁^{m}∥yᵢ−f(xᵢ,ω)∥

where m is size of the learning sample of the neural network,

where yᵢ is the vector of the expected solution at input xᵢ, where f(xᵢ,ω) is the vector of the solution of the network and

where ω is the vector of weights of synaptic links of the neural network.

As the learning algorithm of the neural network, the Levenberg-Marquardt algorithm may be employed, which, in the vector-matrix form, is written as follows:

(J^{T}J+λ·diag(J^{T}J))·η=J^{T}·(y−f(ω))

where J is the Jacobian matrix, whose i-th line is determined as

$${J}_{i} = \frac{\partial }{\partial ω} f ( {x}_{i} , ω ) ,$$

η is current step for ω, and λ is a factor calculated by defined rules at each iteration (e.g., a small value corresponds to iterations close to the Gauss-Newton algorithm, while a large value corresponds to iterations close to the gradient descent method). At slow steps of the learning algorithm, the current step for ω may be calculated using the precise value of Hessian of the error functional Q(ω) (e.g., this is the technique that uses a modification of the traditional Levenberg-Marquardt algorithm):

$${η}_{jh} = \frac{η}{\frac{{\partial }^{2} Q}{\partial {ω}_{jh}^{2}} + μ}$$

where Q(ω) is the error functional,

where ηⱼₕ is current step for weight factor ωⱼₕ,

where η is global parameter of the learning rate, and

where μ is parameter preventing zero-setting of denominator and, respectively, unlimited increase of the step.

The results obtained were are as follows:

(1) error of classification of the patients into groups “healthy”/“diseased” when using standard statistical parameters of the R-R interval was 43.7%, which is somewhat better than when using the random mechanism of the determination of whether a patient belongs to “healthy” or “diseased” groups (e.g., neural network mistakes were made in almost every 2^{nd} case); and

(2) error of classification of the patients into “healthy”/“diseased” groups when using the present invention's parameters of the informational entropy of the beta-distribution was 22.4%. It follows therefore, that when answering the question of whether a patient belongs to the “healthy” or “diseased” group, neural networks employing the present invention's parameters of the informational entropy of the beta-distribution made an error in only each 4^{th}-5^{th} case.

Therefore, the probability of correctly breaking down the patients into “healthy”/“diseased” groups on the basis of VCR data with the use of the prior-art parameters is 56.3%, while with use of the present invention's parameters of the informational entropy of the beta-distribution is 77.6%. Accordingly, there is a significant improvement with the methodology of the present invention as compared to conventional methodologies.

Referring to FIG. 9, there is shown a schematic view of an illustrative computer based system for carrying out the above-described cardiac rhythm variability analysis of the present invention. In this view, raw EKG data is procured from one or more patients. The raw data, whether in the form of electornic files or paper records, is then transmitted, by any means (e.g., via a computer disk, modem, wirelessly, and/or the like), to a computer or other data processor and/or communication device. The raw EKG data may then be stored on the computer or other data processor and/or communication device for intital processing, or alternatively, may be further transmitted to a remote computer (e.g., a network server) or other data processor and/or communication device for initial and/or further processing. Regardless, the raw and/or partially processed EKG data is then finally processed by the appropriate computer or other data processor and/or communication device to produce the processed EKG data, e.g., in the form of an average value of an informational entropy of a beta-distribution of a plurality of R-R intervals of the patient, a difference between a maximum value and a minimum value of the informational entropy of the beta-distribution of the plurality of R-R intervals of the patient, a root-mean-square deviation value of the informational entropy of the beta-distribution of the plurality of R-R intervals of the patient, and/or a variation factor percentage of the informational entropy of the beta-distribution of the plurality of R-R intervals of the patient. This information may then be used to estimate or infer one or more patient's cardiac rhythm variability to determine or classify the patient's cardiac health status along a continuum going from, for example, “Healthy persons from 18 to 23 y/o,” “Healthy persons from 30 to 62 y/o,” “Patients with a diagnosis of “discirculatory encephalopathy,”” and “Patients with a diagnosis of “apoplectic attack” and/or any other appropriate diagnostic scale that may be chosen.

In some applications, the present invention described above may be provided as elements of an integrated software system, in which the features may be provided as separate elements of a computer program. Some embodiments may be implemented, for example, using a computer-readable storage medium (e.g., non-transitory) or article which may store an instruction or a set of instructions that, if executed by a processor, may cause the processor to perform a method in accordance with the embodiments. Other applications of the present invention may be embodied as a hybrid system of dedicated hardware and software components. Moreover, not all of the features described above need be provided or need be provided as separate units. Additionally, it is noted that the arrangement of the features do not necessarily imply a particular order or sequence of events, nor are they intended to exclude other possibilities. For example, the features may occur in any order or substantially simultaneously with each other. Such implementation details are immaterial to the operation of the present invention unless otherwise noted above.

The exemplary methods and computer program instructions may be embodied on a computer readable storage medium (e.g., non-transitory) that may include any medium that can store information. Examples of a computer readable storage medium (e.g., non-transitory) include electronic circuits, semiconductor memory devices, ROM, flash memory, erasable ROM (EROM), floppy diskette, CD-ROM, optical disk, hard disk, fiber optic medium, or any electromagnetic or optical storage device. In addition, a server or database server may include computer readable media configured to store executable program instructions. The features of the embodiments of the present invention may be implemented in hardware, software, firmware, or a combination thereof and utilized in systems, subsystems, components or subcomponents thereof.

Furthermore, a software program embodying the features of the present invention may be used in conjunction with a computer device. Examples of a computing device may include, but are not limited to, an electronic book reading device, a computer workstation, a terminal computer, a server computer, a handheld device (e.g., a tablet computer, a personal digital assistant “PDA”, a mobile telephone, a Smartphone, etc.), a web appliance, a network router, a network switch, a network bridge, any machine capable of executing a sequence of instructions that specify an action to be taken by that machine, and any combinations thereof. In one example, a computing device may include and/or be included in, a kiosk.

The computer device may also include an input device. In one example, a user of the computer device may enter commands and/or other information into computer device via an input device. Examples of an input device may include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), touchscreen, and any combinations thereof. The input device may be interfaced to bus via any of a variety of interfaces including, but not limited to, a serial interface, a parallel interface, a game port, a USB interface, a FIREWIRE interface, a direct interface to bus, and any combinations thereof. The input device may include a touch screen interface that may be a part of or separate from the display.

A user may also input commands and/or other information to the computer device via a storage device (e.g., a removable disk drive, a flash drive, etc.) and/or a network interface device. A network interface device, such as network interface device may be utilized for connecting the computer device to one or more of a variety of networks, such as a network, and one or more remote devices connected thereto. Examples of a network interface device may include, but are not limited to, a network interface card (e.g., a mobile network interface card, a LAN card), a modem, and any combination thereof. Examples of a network may include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software, etc.) may be communicated to and/or from the computer device via a network interface device.

The computer device may further include a video display adapter for communicating a displayable image to a display device, such as a display device. Examples of a display device may include, but are not limited to, a liquid crystal display (LCD), a cathode ray tube (CRT), a plasma display, a light emitting diode (LED) display, and any combinations thereof. In addition to a display device, the computer device may include one or more other peripheral output devices including, but not limited to, an audio speaker, a printer, and any combinations thereof. Such peripheral output devices may be connected to a bus via a peripheral interface. Examples of a peripheral interface may include, but are not limited to, a serial port, a USB connection, a FIREWIRE connection, a parallel connection, and any combinations thereof.

While the invention has been described with reference to an exemplary embodiment, it will be understood by those skilled in the art that various changes can be made and equivalents can be substituted for elements thereof without departing from the scope of the invention. In addition, many modifications can be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A computer based system for cardiac rhythm variability analysis of an electrocardiogram, wherein the electrocardiogram includes a plurality of recorded R-R intervals, comprising:
a computer processor unit; and
a non-transitory computer readable medium operably associated with the computer processor unit, the non-transitory computer readable medium storing instructions executable by the computer processor unit to perform the steps of:
determining an average value of an informational entropy of a beta-distribution of the plurality of R-R intervals;
determining a difference between a maximum value and a minimum value of the informational entropy of the beta-distribution of the plurality of R-R intervals;
determining a root-mean-square deviation value of the informational entropy of the beta-distribution of the plurality of R-R intervals; and
determining a variation factor percentage of the informational entropy of the beta-distribution of the plurality of R-R intervals.

2. The system according to claim 1, wherein the variation factor percentage is determined by subtracting from 100 the result of dividing the root-mean-square deviation value by the average value of the informational entropy of the beta-distribution of the plurality of R-R intervals, wherein the result of dividing the root-mean-square deviation value by the average value of the informational entropy of the beta-distribution of the plurality of R-R intervals is expressed as a positive number.

3. The system according to claim 1, further comprising a display device operably associated with the computer processor unit.

4. The system according to claim 1, further comprising a data input system operably associated with the computer processor unit.

5. The system according to claim 1, further comprising a mobile telecommunications system operably associated with the computer processor unit.

6. The system according to claim 1, further comprising the step of determining a cardiac rhythm status of a patient based on the step of determining the average value of the informational entropy of the beta-distribution of the plurality of R-R intervals of the patient.

7. The system according to claim 1, further comprising the step of determining a cardiac rhythm status of a patient based on the step of determining the difference between the maximum value and the minimum value of the informational entropy of the beta-distribution of the plurality of R-R intervals of the patient.

8. The system according to claim 1, further comprising the step of determining a cardiac rhythm status of a patient based on the step of determining the root-mean-square deviation value of the informational entropy of the beta-distribution of the plurality of R-R intervals of the patient.

10. The system according to claim 1, further comprising the step of determining a cardiac rhythm status of a patient based on the step of determining the variation factor percentage of the informational entropy of the beta-distribution of the plurality of R-R intervals of the patient.

11. A computer implemented method for cardiac rhythm variability analysis, comprising the steps of:
providing a computer processor unit;
providing a non-transitory computer readable medium operably associated with the computer processor unit, the non-transitory computer readable medium storing instructions executable by the computer processor unit to perform the steps of:
determining an average value of an informational entropy of a beta-distribution of the plurality of R-R intervals;
determining a difference between a maximum value and a minimum value of the informational entropy of the beta-distribution of the plurality of R-R intervals;
determining a root-mean-square deviation value of the informational entropy of the beta-distribution of the plurality of R-R intervals; and
determining a variation factor percentage of the informational entropy of the beta-distribution of the plurality of R-R intervals.

12. The system according to claim 11, wherein the variation factor percentage is determined by subtracting from 100 the result of dividing the root-mean-square deviation value by the average value of the informational entropy of the beta-distribution of the plurality of R-R intervals, wherein the result of dividing the root-mean-square deviation value by the average value of the informational entropy of the beta-distribution of the plurality of R-R intervals is expressed as a positive number.

13. The system according to claim 11, further comprising the step of providing a display device operably associated with the computer processor unit.

14. The system according to claim 11, further comprising the step of providing a data input system operably associated with the computer processor unit.

15. The system according to claim 11, further comprising the step of providing a mobile telecommunications system operably associated with the computer processor unit.

16. The system according to claim 11, further comprising the step of determining a cardiac rhythm status of a patient based on the step of determining the average value of the informational entropy of the beta-distribution of the plurality of R-R intervals of the patient.

17. The system according to claim 11, further comprising the step of determining a cardiac rhythm status of a patient based on the step of determining the difference between the maximum value and the minimum value of the informational entropy of the beta-distribution of the plurality of R-R intervals of the patient.

18. The system according to claim 11, further comprising the step of determining a cardiac rhythm status of a patient based on the step of determining the root-mean-square deviation value of the informational entropy of the beta-distribution of the plurality of R-R intervals of the patient.

19. The system according to claim 1, further comprising the step of determining a cardiac rhythm status of a patient based on the step of determining the variation factor percentage of the informational entropy of the beta-distribution of the plurality of R-R intervals of the patient.

20. A computer based system for cardiac rhythm variability analysis of an electrocardiogram, wherein the electrocardiogram includes a plurality of recorded R-R intervals, comprising:
a computer processor unit;
a display device operably associated with the computer processor unit;
a data input system operably associated with the computer processor unit; and
a non-transitory computer readable medium operably associated with the computer processor unit, the non-transitory computer readable medium storing instructions executable by the computer processor unit to perform the steps of:
determining an average value of an informational entropy of a beta-distribution of the plurality of R-R intervals;
determining a difference between a maximum value and a minimum value of the informational entropy of the beta-distribution of the plurality of R-R intervals;
determining a root-mean-square deviation value of the informational entropy of the beta-distribution of the plurality of R-R intervals;
determining a variation factor percentage of the informational entropy of the beta-distribution of the plurality of R-R intervals, wherein the variation factor percentage is determined by subtracting from 100 the result of dividing the root-mean-square deviation value by the average value of the informational entropy of the beta-distribution of the plurality of R-R intervals, wherein the result of dividing the root-mean-square deviation value by the average value of the informational entropy of the beta-distribution of the plurality of R-R intervals is expressed as a positive number; and
determining a cardiac rhythm status of a patient based on any one of the steps of determining the average value of the informational entropy of the beta-distribution of the plurality of R-R intervals of the patient, determining the difference between the maximum value and the minimum value of the informational entropy of the beta-distribution of the plurality of R-R intervals of the patient, determining the root-mean-square deviation value of the informational entropy of the beta-distribution of the plurality of R-R intervals of the patient, or determining the variation factor percentage of the informational entropy of the beta-distribution of the plurality of R-R intervals of the patient.

21. A computer implemented method for cardiac rhythm variability analysis, comprising the steps of:
providing a computer processor unit;
providing a display device operably associated with the computer processor unit;
providing a data input system operably associated with the computer processor unit; and
providing a non-transitory computer readable medium operably associated with the computer processor unit, the non-transitory computer readable medium storing instructions executable by the computer processor unit to perform the steps of:
determining an average value of an informational entropy of a beta-distribution of the plurality of R-R intervals;
determining a difference between a maximum value and a minimum value of the informational entropy of the beta-distribution of the plurality of R-R intervals;
determining a root-mean-square deviation value of the informational entropy of the beta-distribution of the plurality of R-R intervals; and
determining a variation factor percentage of the informational entropy of the beta-distribution of the plurality of R-R intervals, wherein the variation factor percentage is determined by subtracting from 100 the result of dividing the root-mean-square deviation value by the average value of the informational entropy of the beta-distribution of the plurality of R-R intervals, wherein the result of dividing the root-mean-square deviation value by the average value of the informational entropy of the beta-distribution of the plurality of R-R intervals is expressed as a positive number; and
determining a cardiac rhythm status of a patient based on any one of the steps of determining the average value of the informational entropy of the beta-distribution of the plurality of R-R intervals of the patient, determining the difference between the maximum value and the minimum value of the informational entropy of the beta-distribution of the plurality of R-R intervals of the patient, determining the root-mean-square deviation value of the informational entropy of the beta-distribution of the plurality of R-R intervals of the patient, or determining the variation factor percentage of the informational entropy of the beta-distribution of the plurality of R-R intervals of the patient.
